(19) United States
(12) Patent Application Publication
(10) Pub. No.: US 2021/0187233 A1
(43) Pub. Date: Jun. 24, 2021

(54) ADHESIVE SKIN APPLICATION SYSTEM

(71) Applicant: KONINKLIJKE PHILIPS N.V., Eindhoven (NL)

(72) Inventors: Richard Thomas Haibach, Verona, PA (US); Natasha A. Gilbert, Monroeville, PA (US); Lauren Patricia Chodkowski, Monroeville, PA (US)

(21) Appl. No.: 17/110,402

(22) Filed: Dec. 3, 2020

Related U.S. Application Data
(60) Provisional application No. 62/950,571, filed on Dec. 19, 2019.

(51) Int. Cl. A61M 16/06; A61M 16/00; A61M 16/08

(52) U.S. Cl. CPC A61M 16/0688; A61M 16/0875; A61M 16/0057; A61M 16/0666

(57) ABSTRACT

A coupling arrangement for use in coupling a patient interface to the skin of a patient includes an adhesive arrangement and an application system. The adhesive arrangement includes: a substrate material having a first surface and a second surface opposite the first surface, a first adhesive material provided on the first surface, and a second adhesive material provided on the second surface. The application system is disposed on the first adhesive material and includes a number of separate portions, with each portion formed from a film material. Each separate portion includes: a base disposed on at least a portion of the first adhesive material, and a tab coupled to an interior edge of the base and extending upward and away therefrom, the tab being sized and configured to be grasped by the patient at an end portion opposite the interior edge.

### CROSS-REFERENCE TO RELATED APPLICATIONS

This patent application claims the priority benefit under 35 U.S.C. § 119(e) of U.S. Provisional Application No. 62/950,571, filed on Dec. 19, 2019, the contents of which are herein incorporated by reference.

### BACKGROUND OF THE INVENTION

### 1. Field of the Invention

The present invention relates to systems for securing a patient interface, such as a nasal mask, with an adhesive arrangement to the face of a patient. More particularly, the present invention relates to a system for use in aligning and applying an adhesive arrangement to a patient interface and for aligning and adhering the adhesive arrangement along with the patient interface to the skin of the patient. The present invention also relates to methods of using the aforementioned system.

### 2. Description of the Related Art

Many individuals suffer from disordered breathing during sleep. Sleep apnea is a common example of such sleep disordered breathing suffered by millions of people throughout the world. One type of sleep apnea is obstructive sleep apnea (OSA), which is a condition in which sleep is repeatedly interrupted by an inability to breathe due to an obstruction of the airway; typically the upper airway or pharyngeal area. Obstruction of the airway is generally believed to be due, at least in part, to a general relaxation of the muscles which stabilize the upper airway segment, thereby allowing the tissues to collapse the airway. Another type of sleep apnea syndrome is a central apnea, which is a cessation of respiration due to the absence of respiratory signals from the brain's respiratory center. An apnea condition, whether obstructive, central, or mixed, which is a combination of obstructive and central, is defined as the complete or near cessation of breathing, for example a 90% or greater reduction in peak respiratory air-flow.

Those afflicted with sleep apnea experience sleep fragmentation and complete or nearly complete cessation of ventilation intermittently during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of sleep apnea include right ventricular dysfunction, carbon dioxide retention during wakefulness, as well as during sleep, and continuous reduced arterial oxygen tension. Sleep apnea sufferers may be at risk for excessive mortality from these factors as well as by an elevated risk for accidents while driving and/or operating potentially dangerous equipment.

Even if a patient does not suffer from a complete or nearly complete obstruction of the airway, it is also known that adverse effects, such as arousals from sleep, can occur where there is only a partial obstruction of the airway. Partial obstruction of the airway typically results in shallow breathing referred to as a hypopnea. A hypopnea is typically defined as a 50% or greater reduction in the peak respiratory air-flow. Other types of sleep disordered breathing include, without limitation, upper airway resistance syndrome (UARS) and vibration of the airway, such as vibration of the pharyngeal wall, commonly referred to as snoring.

It is well known to treat sleep disordered breathing by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's breathing effort, to increase the comfort to the patient. This pressure support technique is referred to as bi-level pressure support, in which the inspiratory positive airway pressure (IPAP) delivered to the patient is higher than the expiratory positive airway pressure (EPAP). It is further known to provide a positive pressure therapy in which the pressure is automatically adjusted based on the detected conditions of the patient, such as whether the patient is experiencing an apnea and/or hypopnea. This pressure support technique is referred to as an auto-titration type of pressure support, because the pressure support device seeks to provide a pressure to the patient that is only as high as necessary to treat the disordered breathing.

Pressure support therapies as just described involve the placement of a patient interface device including a mask component having a soft, flexible sealing cushion member on the face of the patient. The mask component may be, without limitation, a nasal mask that covers the patient's nose, a nasal/oral mask that covers the patient's nose and mouth, or a full face mask that covers the patient's face. Such patient interface devices may also employ other patient contacting components, such as forehead supports, cheek pads and chin pads. The patient interface device is connected to a gas delivery tube or conduit and interfaces the pressure support device with the airway of the patient, so that a flow of breathing gas can be delivered from the pressure/flow generating device to the airway of the patient.

Traditionally, such patient interface devices have been secured to the face/head of the patient by a headgear component having one or more straps which wrap around all, or a portion, of the patient's head. Recently, adhesive arrangements have been employed, either in-whole, or in-part, to secure patient interface devices to the face of a patient. In such arrangements, many concerns/complaints are related to the strength of the adhesive and the ease of removal of the adhesive. Another major concern/complaint is the ability to apply the mask perfectly the first time. Currently nothing on the market allows for an easy alignment to the face or ease of readjustment without having the remove the adhesive. Many people notice skin irritation when having to remove the adhesive many times in a short period due to application failure. Application error can also cause nasal occlusion, pressure points, and an overall discomfort.

### SUMMARY OF THE INVENTION

Accordingly, as one aspect of the present invention a coupling arrangement for use in coupling a patient interface to the skin of a patient is provided. The coupling arrangement comprises: an adhesive arrangement comprising: a substrate material having a first surface and a second surface opposite the first surface, a first adhesive material provided on the first surface, and a second adhesive material provided on the second surface; and an application system disposed on the first adhesive material, the application system comprising: a number of separate portions, each portion formed from a film material and including: a base disposed on at least a portion of the first adhesive material, and a tab coupled to an interior edge of the base and extending upward and away therefrom, the tab being sized and configured to be grasped by the patient at an end portion opposite the interior edge.

For each separate portion, the base and the tab may be formed from a single unitary piece of the film material separated by a fold.

The number of separate portions may comprise a plurality of separate portions.

The adhesive arrangement may comprise a pair of apertures defined therein.

The adhesive arrangement may further comprise a layer of a release film provided covering the second adhesive material.

As another aspect of the present invention, an airway pressure support system for use in providing a flow of a breathing gas to the airway of a patient is provided. The pressure support system comprises: a patient interface structured to engage the face of the patient about the airway of the patient; and a coupling arrangement comprising and adhesive arrangement as described herein, wherein the second surface of the substrate material of the adhesive arrangement is coupled to a surface of the patient interface via the second adhesive material.

The patient interface may comprise a cradle-like nasal interface structured to engage the underside of the nose of the patient.

The surface of the patient interface may comprise a contoured surface, the patient interface may comprise a pair of nasal apertures defined in the contoured surface, the adhesive arrangement may comprise a pair of apertures defined therein, and each aperture of the adhesive arrangement may be aligned with a corresponding nasal aperture of the patient interface.

The airway pressure support system may further comprise: a gas flow generator that is structured to generate the flow of the breathing gas; and a hose having a first end coupled to the gas flow generator and an opposite second end coupled to the patient interface, wherein the hose is structured to convey the flow of the breathing gas from the gas flow generator to the patient interface.

As yet another aspect of the present invention, a method of coupling a patient interface to the skin of a patient via a coupling arrangement as described herein is provided. The method comprises: adhering the coupling arrangement to a surface of the patient interface via the second adhesive material; urging the patient interface assembly toward the skin of the patient such that each tab is compressed between the skin of the patient and the adhesive arrangement with each end portion of each tab extending outward from the patient interface; and pulling the end portion of the tab of one portion of the number of separate portions outward so as to expose a portion of the first adhesive material and adhering the skin of the patient thereto.

The method may further comprise: pulling the end portion of the tab of another portion of the number of separate portions outward so as to expose a another portion of the first adhesive material and adhering the skin of the patient thereto.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a simplified perspective front view of an airway pressure support system including a patient interface along with a coupling arrangement, in accordance with one non-limiting example embodiment of the present invention, shown with components thereof exploded;

FIG. 2 is a rear elevation view of the patient interface assembly of FIG. 1 shown with the adhesive arrangement and application system of FIG. 1 disposed thereon;

FIG. 3 is a top view of the arrangement of FIG. 2 with the application system shown in dashed line so as to show the general relationship with the adhesive arrangement and patient interface assembly below;

FIG. 4 is an elevation section view of the arrangement of FIGS. 2 and 3 taken along line 4-4 of FIG. 3;

FIG. 5 is a top view, similar to that of FIG. 3, except showing (in dashed line) another application system in accordance with another one example embodiment of the present invention; and

FIGS. 6A-6C show the arrangement of FIGS. 1 and 4 positioned against the nose of a patient illustrating steps in securing the patient interface of FIG. 1 to the nose of a patient using the application system.

### DETAILED DESCRIPTION OF EXEMPLARY EMBODIMENTS

As required, detailed embodiments of the present invention are disclosed herein; however, it is to be understood that the disclosed example embodiments described herein are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are coupled directly in contact with each other. As used herein, “fixedly coupled” or “fixed” means that two components are coupled so as to move as one while maintaining a constant orientation relative to each other.

As used herein, the statement that two or more parts or components “engage” one another shall mean that the parts exert a force against one another either directly or through one or more intermediate parts or components. As used herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

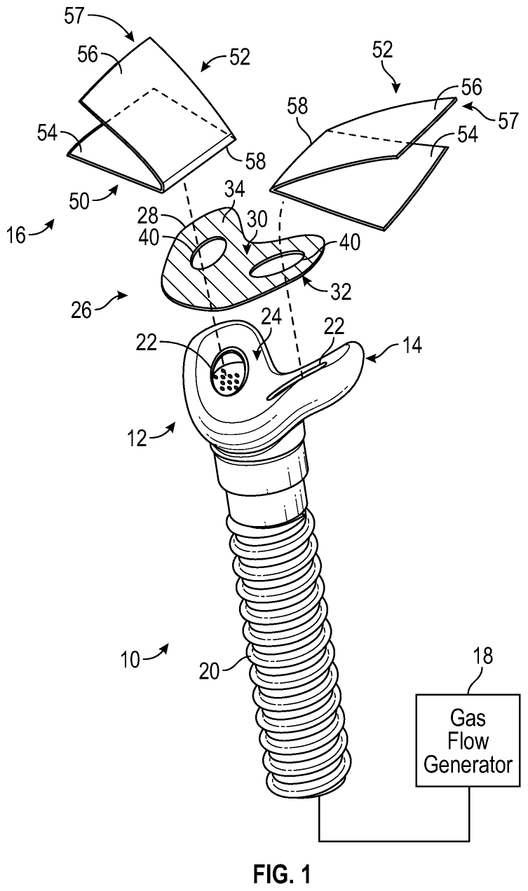

FIG. 1 is a simplified perspective front view of an airway pressure support system 10 for use in providing a flow of a breathing gas to the airway of a patient (not shown). Airway pressure support system 10 includes a patient interface assembly 12 having a patient interface 14 and a coupling arrangement 16 (shown exploded) for use in coupling patient interface 14 to the face of the patient. Airway pressure support system 10 includes a gas flow generator 18 (shown schematically) and a hose 20 (partially shown schematically) having a first end (not numbered) coupled to gas flow generator 18 and an opposite second end (not numbered) coupled to patient interface 14. Gas flow generator 18 is structured to generate a flow of breathing gas to be delivered to an airway of a patient via hose 20 and patient interface 14.

In the example arrangement shown in FIG. 1, patient interface 14 is in the form of a cradle-like nasal interface for generally engaging the underside of the nose of a patient. Accordingly, patient interface 14 includes a pair nasal apertures 22 defined in a contoured surface 24. In one example embodiment of the present invention, contoured surface 24 and each nasal aperture 22 is custom dimensioned/formed based on facial dimensions of a particular patient. Such custom dimensioning/forming may readily be carried out using 3D scanning and printing techniques known in the art. It is to be appreciated however, that other arrangements of patient interface 14 may be employed without varying from the scope of the present invention.

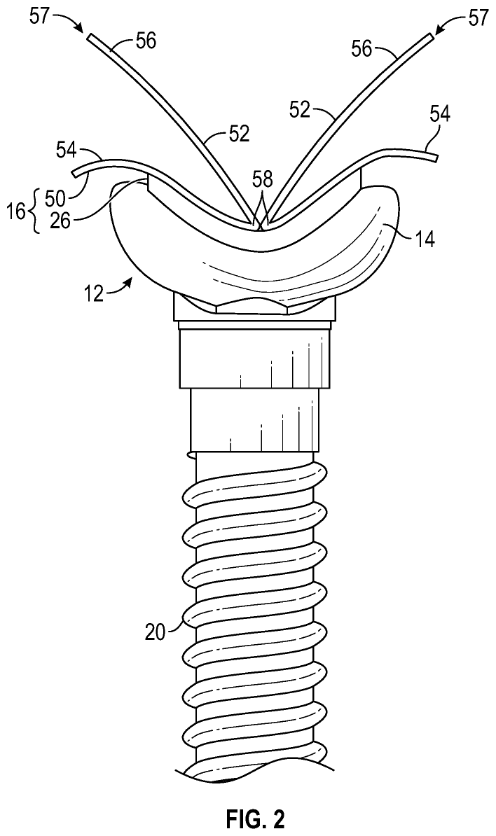

Continuing to refer to FIG. 1, coupling arrangement 16 includes an adhesive arrangement 26 used for securing patient interface 14 to the face of a patient. Adhesive arrangement 26 is formed from a generally thin (e.g., having a thickness of at least 0.04 mm) substrate material 28 having a first surface 30, which faces toward a patient (i.e., upward in FIG. 1), and a second surface 32, opposite first surface 30, which faces toward patient interface 14 (i.e., downward in FIG. 1). Planar substrate material 28 may generally be any pliable material such as, for example, without limitation, foam, silicone polyurethane, latex, or any other suitable material. Adhesive arrangement 26 further includes a first adhesive material 34 (shown schematically as hatching, e.g., without limitation, a silicone or acrylic based adhesive layer or any other suitable adhesive layer) provided on first surface 30 for use in adhering adhesive arrangement 26 to the skin of a patient. Similarly, adhesive arrangement 26 also includes a second adhesive material (not numbered) provided on second surface 32 for use in adhering adhesive arrangement 26 to contoured surface 24 of patient interface 14, such as shown in FIGS. 2-4, 5A-5C and 6.

In order to provide for a reliable seal about each n are of the patient, and to allow for the passage of the flow of breathing gas from each of nasal apertures 22 of patient interface 14 to the respective nares of the patient, adhesive arrangement 26 includes a pair of apertures 40 defined therein of similar, and preferably identical shape (e.g., to minimize turbulence, maximize comfort, etc.), as nasal apertures 22.

As discussed further below, in one example embodiment of the present invention adhesive arrangement 26 is a one-time use item that is used in securing patient interface 14 to the skin on the underside of the nose of a patient. As patient interface 14 is intended to be used multiple times, a new adhesive arrangement 26 would be adhered to patient interface 14 before each use. In such embodiment, adhesive arrangement 26 further include a layer of release film (not numbered) provided covering second adhesive material on second surface 32 for the second adhesive material until desired to be adhered to contoured surface 24 of patient interface 24.

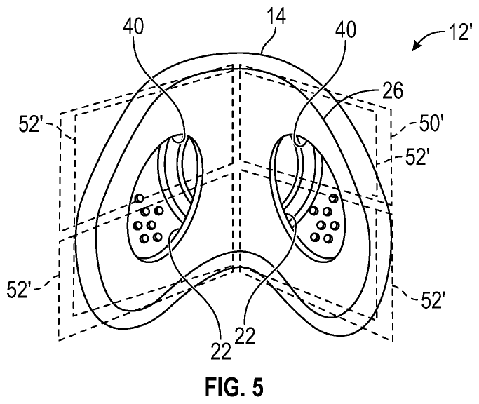

Continuing to refer to FIG. 1, as well as to FIGS. 2-4, coupling arrangement 16 further includes an application system 50 disposed on first adhesive material 34 of adhesive arrangement 26. Application system 50 includes a number of separate portions 52. In the one example embodiment shown in FIGS. 1-4, application system 50 includes two portions 52, however, it is to be appreciated that the quantity of portions may be varied without varying from the scope of the present invention. For example, FIG. 5 shows another patient interface assembly 12′ in accordance with one example embodiment of the present invention that is similar to the example shown in FIGS. 1-4 except such embodiment utilizes an application system 50′ including four separate portions 52′.

Referring again to FIGS. 1-4, each portion 52 of application system 50 is formed separately from a suitable release film and is of similar design, hence only one portion 52 will be described in detail herein. Each portion 52 includes a base 54 and a tab 56. Base 54 is sized and configured to be disposed covering at least a portion of first adhesive material 34 of adhesive arrangement 26. Tab 56 is coupled to an edge of base 54 and extends upward and away from the base. In the example shown in FIGS. 1-4, tab 56 extends from an interior edge of base 54. Tab 56 is sized and configured to be grasped by a patient, e.g., between a thumb and forefinger, at an end portion 57 opposite the interior edge as discussed below. In the one example shown in FIGS. 1-4, base 54 and tab 56 are formed as portions of a single unitary piece of material that have generally been delineated by a fold 58, however, it is to be appreciated that base 54 and tab 56 may be formed separately and subsequently coupled without varying from the scope of the present invention.

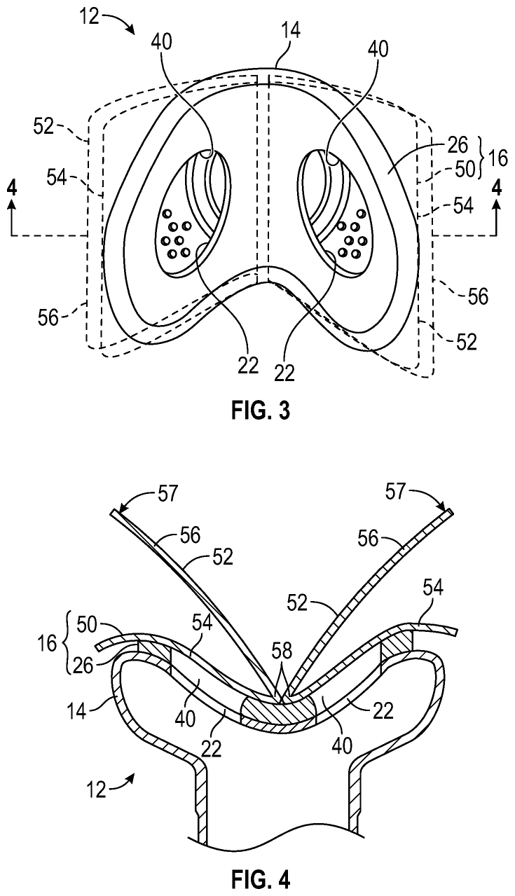

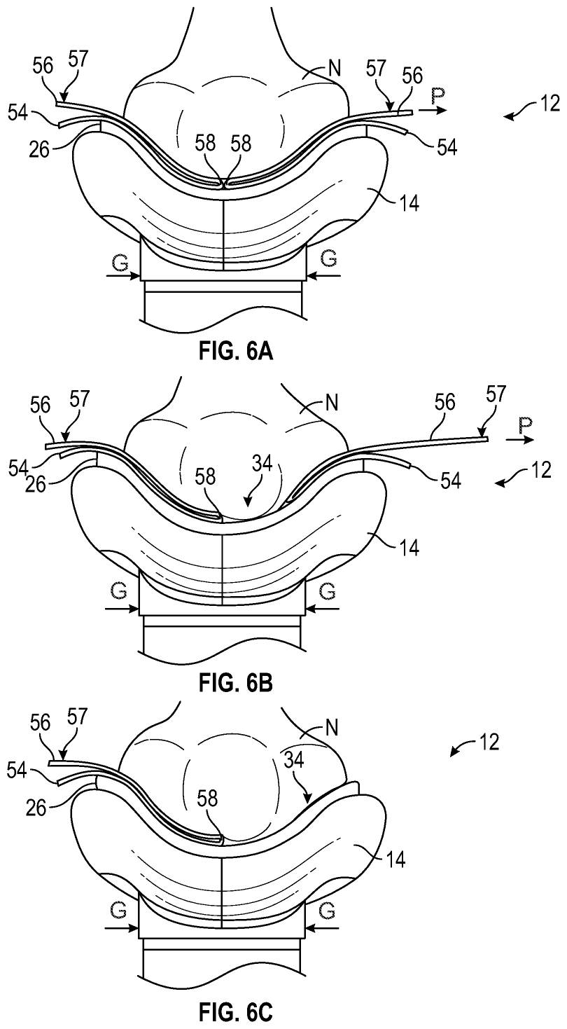

Having thus described the general arrangement and components of one example patient interface assembly 12, a method of coupling patient interface 14 thereof to a nose N of a patient using coupling arrangement 16 will now be discussed in conjunction with FIGS. 6A-6C. In such views, coupling arrangement 16 was previously installed on patient interface 14 and adhered to contoured surface 24 thereof via the second adhesive (after removing the release film therefrom) of coupling arrangement 16. Coupling arrangement 16 is aligned with patient interface 14 such that apertures 40 thereof are aligned with nasal apertures 22 of patient interface 14 (e.g., such as shown in FIG. 3). One or more of tabs 56 of application system 50 may be utilized in gripping coupling arrangement 16 during such alignment and adhering to patient interface 14.

Referring now to FIG. 6A, patient interface assembly 12 is urged upward toward the underside of a patient's nose N such that each tab 56 is compressed between the underside of nose N and adhesive arrangement 26 with each end portion 57 of each tab 56 extending outward from nose N. Such positioning may be accomplished by the patient grasping patient interface 14 by applying forces such as indicated by arrows G, such as by grasping with the thumb and forefinger of the right hand of the patient. Patient interface 14 may readily be positioned by the patient with respect to nose N as portions 50 cover first adhesive 34 of adhesive arrangement 26.

Referring now to FIG. 6B, adherence of nose N to adhesive arrangement 16 via first adhesive 34 thereof begins by gripping end portion 57 of tab 56 of one of portions 52, and pulling outward such as shown by each arrow P. Such gripping of end portion 57 may be carried out by the patient grasping with the thumb and forefinger of the left hand of the patient. As end portion 57 and thus tab 56 is pulled outward, base 54 is generally rolled/folded back onto itself, thus slowly exposing first adhesive 34 of adhesive arrangement 16 for adherence to the skin of the underside of nose N. As first adhesive 34 is slowly exposed, small adjustments of patient interface 14 relative to nose N may generally be made without discomfort to the patient or hampering the adherence of first adhesive 34.

As shown in FIG. 6C, once one portion 52 of alignment system 50 has been removed, half of the underside of nose N is adhered to patient interface 14 via first adhesive 34 of adhesive arrangement 26. The other portion 52 is then removed in a similar manner to fully adhere patient interface 14 to the underside of nose N.

From the foregoing, it is thus to be appreciated embodiments of the present invention provide for alignment systems and coupling arrangements for use in coupling a patient interface to a patient that improve upon existing arrangements.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A coupling arrangement for use in coupling a patient interface to the skin of a patient, the coupling arrangement comprising:
(a) an adhesive arrangement comprising:
(1) a substrate material having a first surface and a second surface opposite the first surface,
(2) a first adhesive material provided on the first surface, and
(3) a second adhesive material provided on the second surface; and
(b) an application system disposed on the first adhesive material, the application system comprising:
a number of separate portions, each portion formed from a film material and including:
(i) a base disposed on at least a portion of the first adhesive material, and
(ii) a tab coupled to an interior edge of the base and extending upward and away therefrom, the tab is sized and configured to be grasped by the patient at an end portion opposite the interior edge.

2. The coupling arrangement of claim 1, wherein for each separate portion, the base and the tab are formed from a single unitary piece of the film material separated by a fold.

3. The coupling arrangement of claim 1, wherein the number of separate portions comprises a plurality of separate portions.

4. The coupling arrangement of claim 1, wherein the adhesive arrangement comprises a pair of apertures defined therein.

5. The coupling arrangement of claim 1, wherein the adhesive arrangement further comprises a layer of a release film provided covering the second adhesive material.

6. An airway pressure support system for use in providing a flow of a breathing gas to the airway of a patient, the pressure support system comprising:
(a) a patient interface structured to engage the face of the patient about the airway of the patient; and
(b) a coupling arrangement comprising:
(1) an adhesive arrangement comprising:
(i) a substrate material having a first surface and a second surface opposite the first surface,
(ii) a first adhesive material provided on the first surface, and
(iii) a second adhesive material provided on the second surface; and
(2) an application system disposed on the first adhesive material, the application system comprising:
a number of separate portions, each portion formed from a film material and including:
a base disposed on at least a portion of the first adhesive material, and
a tab coupled to an interior edge of the base and extending upward and away therefrom, the tab is sized and configured to be grasped by the patient at an end portion opposite the interior edge, wherein the second surface of the substrate material of the adhesive arrangement is coupled to a surface of the patient interface via the second adhesive material.

7. The airway pressure support system of claim 6, wherein the patient interface comprises a cradle-like nasal interface structured to engage the underside of the nose of the patient.

8. The airway pressure support system of claim 6, wherein for each separate portion, the base and the tab are formed from a single unitary piece of the film material separated by a fold.

9. The airway pressure support system of claim 6, wherein the number of separate portions comprises a plurality of separate portions.

10. The airway pressure support system of claim 6, wherein the surface of the patient interface comprises a contoured surface, wherein the patient interface comprises a pair of nasal apertures defined in the contoured surface, wherein the adhesive arrangement comprises a pair of apertures defined therein, and wherein each aperture of the adhesive arrangement is aligned with a corresponding nasal aperture of the patient interface.

11. The airway pressure support system of claim 6, further comprising:
a gas flow generator structured to generate the flow of the breathing gas; and
a hose having a first end coupled to the gas flow generator and an opposite second end coupled to the patient interface, wherein the hose is structured to convey the flow of the breathing gas from the gas flow generator to the patient interface.

12. A method of coupling a patient interface to the skin of a patient via a coupling arrangement having: an adhesive arrangement comprising: a substrate material having a first surface and a second surface opposite the first surface, a first adhesive material provided on the first surface, and a second adhesive material provided on the second surface; and an application system disposed on the first adhesive material, the application system comprising: a number of separate portions, each portion formed from a film material and including: a base disposed on at least a portion of the first adhesive material, and a tab coupled to an interior edge of the base and extending upward and away therefrom, the tab is sized and configured to be grasped by the patient at an end portion opposite the interior edge, the method comprising:
adhering the coupling arrangement to a surface of the patient interface via the second adhesive material;
urging the patient interface assembly toward the skin of the patient such that each tab is compressed between the skin of the patient and the adhesive arrangement with each end portion of each tab extending outward from the patient interface; and
pulling the end portion of the tab of one portion of the number of separate portions outward so as to expose a portion of the first adhesive material and adhering the skin of the patient thereto.

13. The method of claim 12, further comprising: pulling the end portion of the tab of another portion of the number of separate portions outward so as to expose a another portion of the first adhesive material and adhering the skin of the patient thereto.
